(19) United States
(12) Patent Application Publication
(10) Pub. No.: US 2005/0279853 A1
(43) Pub. Date: Dec. 22, 2005

(54) Refill for an air freshener or insecticide device

(76) Inventors: Andrew McLeisch, Sheffield (GB); Clare Louise Jackson, Hull (GB); Shaun Patrick Rymer, Hull (GB)

Correspondence Address: NORRIS, MCLAUGHLIN & MARCUS, 875 THIRD AVE, 18TH FLOOR, NEW YORK, NY, 10022, US

(21) Appl. No.: 10/495,716

(22) PCT Filed: Nov. 19, 2002
(86) PCT No.: PCT/GB02/05189

§ 371 Date: Feb. 14, 2005

Related U.S. Application Data
(63) Continuation-in-part of application No. 09/989,294, filed on Nov. 20, 2001, now abandoned.

(51) Int. Cl. A61L 9/04

(52) U.S. Cl. 239/43; 239/37; 239/53

(57) ABSTRACT

The invention concerns a refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded along at least one fold line which, in use, extends substantially vertically to at least partially surround the cartridge, the sheet of material and cartridge being supported in a frame.

The present invention relates to a refill for a liquid air freshener or insecticidal device.

Refills are known in the art which comprise a cartridge containing a volatile liquid composition to be evaporated and a porous pad positioned, in use, beneath a lower face of the cartridge to receive the liquid composition as it is slowly released from the cartridge.

The present invention provides an improvement to such a refill. According to a first aspect of the present invention, there is provided a refill for a liquid air freshener or insecticidal device comprising a cartridge containing a volatile liquid composition to be evaporated and a porous pad positioned, in use, to receive the liquid composition as it is slowly released under gravity from the cartridge; wherein the porous pad is a part of a sheet of material which is folded to at least partially surround the cartridge.

Such an arrangement provides a simple way of securing the porous pad reliably in position beneath the cartridge which does not require a separate fitting operation by the consumer. It also provides a more substantial package which can be easily handled by the consumer and readily located with the housing of the air freshener or insecticidal device.

According to a second aspect of the present invention, there is provided a refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded along at least one fold line which, in use, extends substantially vertically to at least partially surround the cartridge, the sheet of material and cartridge being supported in a frame.

The provision of a pad with vertical fold lines has the advantage that the assembly of the sheet of material and frame is simply carried out by folding the sheet of material on a mandrel which folded material is then inserted into the frame. Complementary tags may be provided on the sheet and frame in order to guarantee precise location of the sheet within the frame. The folded sheet is retained in the frame by the resilience of the folded structure.

Preferably, the sheet of material comprises the porous pad, a rear panel and two side panels which are folded inwardly along the vertical fold lines towards one another from opposite sides of the rear panel. The porous pad may either be foldably attached to a bottom edge of the rear panel, or may be attached to one or both bottom edges of the side panels along a fold line which, in use, is substantially horizontal.

The frame and sheet may be provided as a sub-assembly wherein the frame has a foldable configuration with at least some of the fold lines corresponding to fold lines on the sheet. Alternatively, however, the frame has a non-foldable configuration as this lends itself to the manufacturing method with the mandrel described above.

According to a third aspect of the present invention there is provided a refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded to at least partially surround the cartridge, the sheet of material and cartridge being supported in a frame; wherein the sheet of material is folded to meet the cartridge towards the end of the cartridge opposite to the end from which the composition is released.

By folding the sheet of material to meet the top end of the cartridge, the stability of the refill structure is enhanced as this top part supports the pad against the cartridge, and also can provide support for side panels of the sheet if these are present.

According to a fourth aspect of the present invention there is provided a refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded to at least partially surround the cartridge, the sheet of material and cartridge being supported in a frame; wherein the cartridge is retained in at least one pair of resilient jaws in the frame.

Providing a pair of resilient jaws to receive the cartridge provides a secure means of retaining the cartridge on the frame. Further, the manufacturing process is simplified as the cartridge is simply required to be moved in a single direction to ensure that it is retained on the frame. Preferably two pairs of resilient jaws are provided spaced along the length of the cartridge.

The present invention also extends to a method of manufacturing a refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded to at least partially surround the cartridge, the sheet of material and cartridge being supported in a frame; the method comprising the steps of: folding the porous pad over a mandrel; removing the mandrel from the folded pad; and inserting the folded pad into the frame, so that the folded pad is retained within the frame by the resilience of the folded structure.

Such a method provides a way of assembling the sheet of material into the frame which simply requires a two-stage process: folding on a mandrel and inserting into the frame. Preferably the frame is formed in a non-foldable configuration.

Preferably, the method further comprises the step of inserting the cartridge into the frame from the opposite direction to the direction in which the pad is inserted on the mandrel. This again facilitates the assembly procedure as the mechanisms for inserting the sheet of material and cartridge allow both to be inserted independently without interference from the other.

Preferably, the cartridge is retained in at least one pair of resilient jaws in the frame. Again, this facilitates insertion of the cartridge in a single direction.

This aspect of the method forms an independent aspect of the present invention which can broadly be defined as a method of making a refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded to at least partially surround the cartridge, the sheet of material and cartridge being supported in a frame; the method comprising the steps of: inserting the folded pad into the frame in a first direction; and inserting the cartridge into the frame in a second direction in which is opposite to the first direction.

Three examples of refills constructed in accordance with the present invention will now be described with reference to the accompanying drawings, in which:

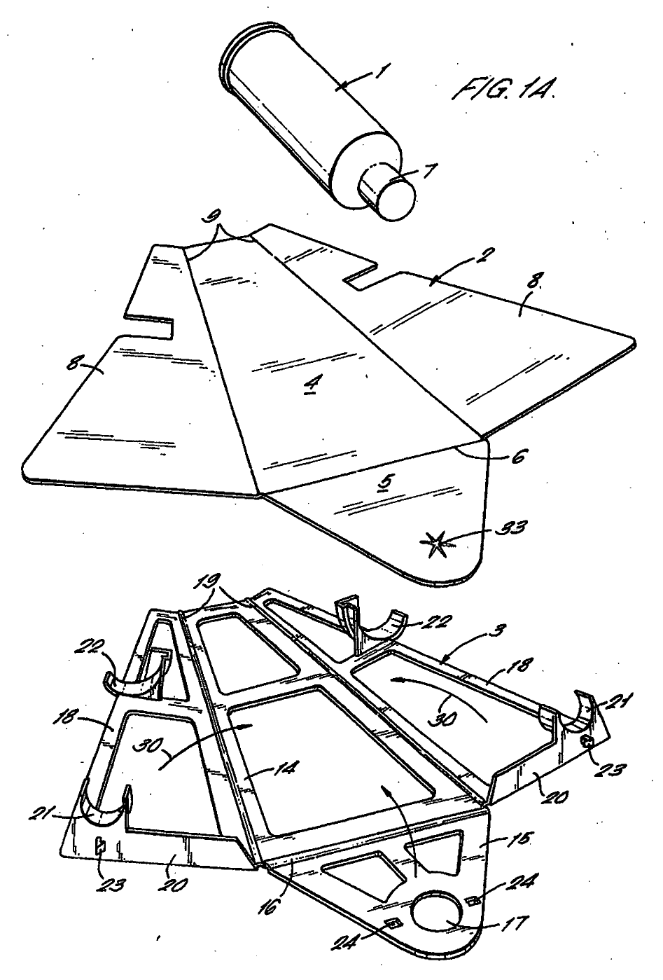

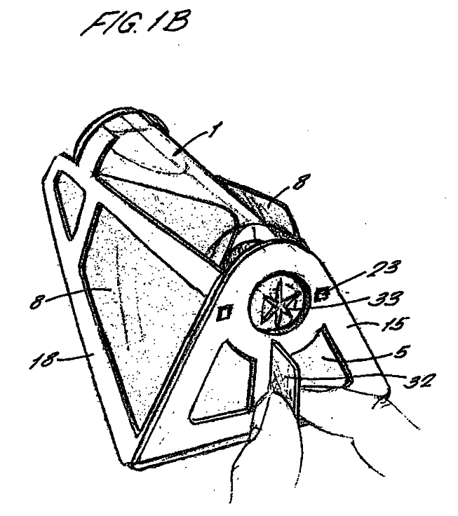

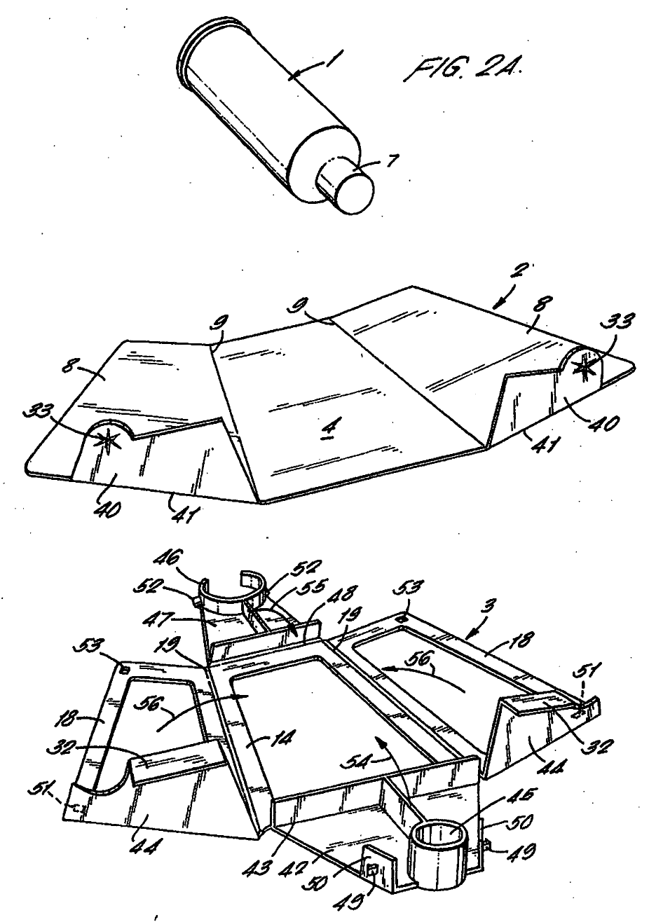

FIG. 1A is an exploded view of a first example showing the assembly of the refill;

FIG. 1B is a perspective view of the assembled refill;

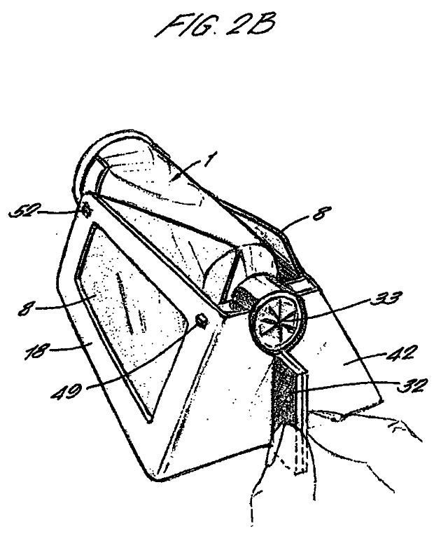

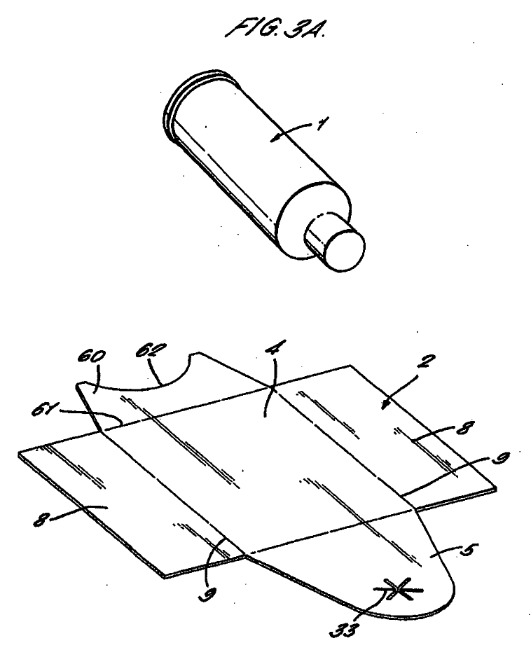

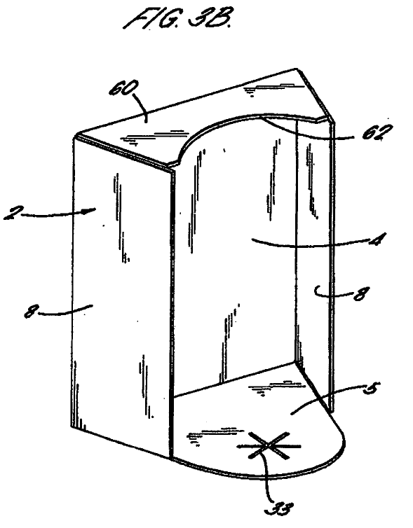

FIG. 2A is a view similar to FIG. 1A of a second example;

FIG. 2B is a view similar to FIG. 1B of a second example;

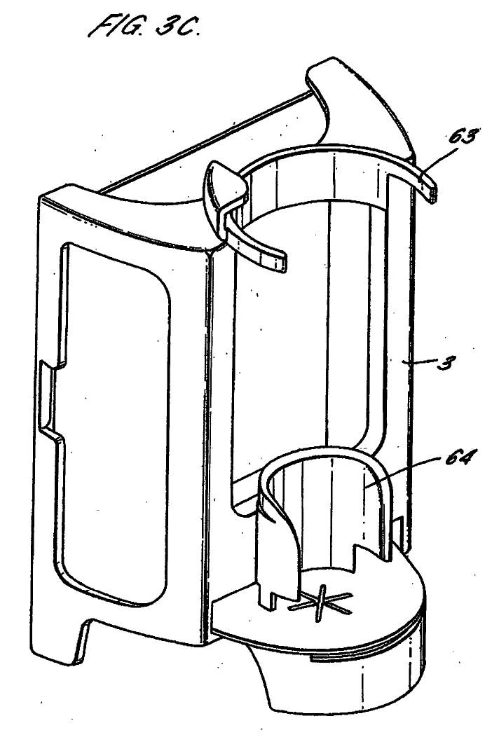

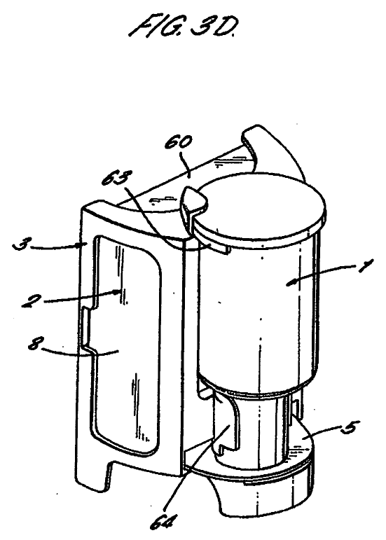

FIGS. 3A and D are similar views to FIGS. 1A and 1B of a third example;

FIG. 3B is a perspective view of the folded sheet of the third example of FIG. 3A; and

FIG. 3C is a view similar to FIG. 3D without the cartridge.

The first example will now be described with reference to FIGS. 1A and 1B.

The refill comprises three components, a cartridge 1, a cardboard sheet 2, and a plastic frame 3.

The sheet 2 comprises a rear panel 4 with a base panel 5 attached along a fold line 6 to its lowermost edge. The base panel 5 provides the porous pad onto which, in use, liquid composition is gradually dispensed from the lower end 7 of cartridge 1. Two sheet side panels 8 are attached via fold lines 9 along the side edges of the rear panel 4.

The sheet 2 sits on the frame 3 which has a shape which generally conforms to the shape of the sheet 2. Thus, the frame 3 has a rear panel 14 corresponding in shape to the rear panel 4 of the sheet and a base panel 15 corresponding in shape to the base panel 5 of the sheet and attached to the rear panel 14 of the base along a fold line 16. The base panel 15 has an aperture 17 positioned to allow penetration of the lower end 7 of the cartridge 1. Two side panels 18 are attached along opposite sides of the rear panel 14 along fold lines 19. An upstanding flange 20 projects from the bottom edge of each side panel 18. A cartridge engaging jaw member 21 projects from the outer edge of the upstanding flange 20. A second jaw member 22 is provided towards the top of each side panel 18. A clip 23 extends from a downwardly facing surface of the upstanding flange 20. The clips 23 are arranged to engage in through holes 24 in the base panel 15.

To assemble the refill, the sheet 2 is positioned on the frame 3. The side panels 8/18 are then folded inwardly in the direction of arrows 30 until the cartridge 1 is held by jaw members 21 and 22. The base panel 5/15 is then folded upwardly in the direction of arrow 31 so that the clips 23 clip into the holes 24, and so that the lower end 7 of cartridge 1 is aligned with the aperture 17, thereby forming the finished refill as shown in FIG. 1B. The clips 23 penetrate the base panel 5 to assist in securing the sheet 2 in place.

As shown in FIG. 1B, a tab 32 is provided on the lower surface of the base panel 15 to allow the refill to be removed from the device housing. Further, a number of slits form a star-shaped aperture 33 in the base panel 5. This aperture 33 is arranged to be pierced by a spike in the housing into which the refill is placed. The spike also pierces the lower end 7 of the cartridge 1 to allow flow of the composition out of the cartridge 1 thereby initiating operation of the device.

The second example will now be described with reference to FIGS. 2A and 2B. Broadly, this has the same construction as the first example with the cartridge 1, cardboard sheet 2 and plastic frame 3. The sheet 2 has a similar rear panel 4 and two adjacent side panels 8 joined to the rear panel along fold lines 9. The frame 3 has a similar rear panel 14 and adjacent side panels 18 joined to the rear panel along fold lines 19.

However, in place of the base panel 5 of the first example, the sheet 2 is provided with a pair of base panels 40, each base panel being attached along a fold line 41 to a lowermost edge of a respective side panel 8. The base panels 40 are half the size of the base panel 5 of the first example and are arranged to meet one another when the refill is folded up to provide a similar configuration to the base panel 5 of the first example. Each base panel 40 has a portion with the star-shaped orifice 33. In the assembled refill, these portions will overlap forming a porous pad of double thickness beneath the lower end 7 of the cartridge 1.

The frame 3 has a base panel 42 attached to the lowermost edge of the rear panel 14 via a fold line 43. Additional parts of the base are provided by a pair of half panels 44 which extend from the lowermost edges of respective side panels 18 and are arranged to support the base panels 40 of the sheet 2. Each half panel 44 has a tab 32 positioned so that tabs 32 abut one another in the assembled refill as shown in FIG. 2B.

The jaw member 21 of the first example is replaced by a cylindrical orifice 45 which is open at its bottom end as shown in FIG. 2B to allow the spike to penetrate when the refill is inserted into the housing. The upper jaw members 22 of the first example are replaced by a single jaw member 46 which is a part circular jaw member attached to the top of an upper panel 47 which is attached to the upper edge of the rear panel 14 along a fold line 48.

To clip the frame together, a pair of clips 49 are provided on flanges 50 which extend upwardly from an outer edge of the base panel 42. These engage with corresponding openings 51 in the side panels 18. A second pair of clips 52 are provided on the top panel 47. These engage with a second pair of openings 53 in the side panels 18.

With the sheet 3 in place on the frame 3, the base panel 42 is folded up in the direction of arrow 54 so that the bottom end of cartridge 1 is retained in cylindrical orifice 45. The top panel 47 is folded downwardly in the direction of arrow 55 so that the top of the cartridge 1 is retained within jaw member 46. The side panels 18 are then folded inwardly in the direction of arrows 56 bringing the tabs 32 together, and bringing the star-shaped orifices 33 into overlapping relation beneath the lower end 7 of the cartridge 1. At the same time, the clips 49 and 52 clip into their respective holes 51 and 53 securing the frame in place. The clips penetrate through the cardboard sheet 2 and also serve to secure this in place. The assembled refill can then be handled as described with reference to the first example.

The third example will now be described with reference to FIGS. 3A, 3B and 3C. Again this has broadly the same construction as the first and second examples with the cartridge 1, cardboard sheet 2 and plastic frame 3. The sheet 2 has a similar rear panel 4 and two adjacent side panels 8 joined to the rear panel along fold lines 9. The sheet 2 has a similar base panel 5 to that of the first example, and in addition has a single end panel 60 which is attached to the rear panel 4 along the fold line 61. The end panel 60 has an arcuate cut out 62, to accommodate the outer wall of the cartridge 1, when the sheet is in position within the frame 3.

The frame 3 differs from the frame of the second example in that it is a non-foldable frame which is not provided in an assembleable form. It is manufactured as a moulding in a preassembled form as shown in FIG. 3C. The sheet 2 is folded on a mandrel along each of the fold lines to form the structure as shown in FIG. 3B. The mandrel is a metal block with an end face shaped generally in the shape of the rear panel 4. The mandrel is then removed from the folded sheet manually and the folded sheet is inserted manually into an open underside of the frame 3. The resilience of the folded sheet retains it in place in the frame. The base panel 5 is supported by the frame 3 in a position such that the lower end of the cartridge 7 will rest on or very close thereto, when it is inserted in the frame 3. The cartridge 1 is inserted into the frame 3 so that it is supported by clip members 63, 64 at each end of the frame 3 in the correct position relative to the base panel 5. The assembled refill can then be handled as described above, with reference to the first example.

1. A refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded along at least one fold line which, in use, extends substantially vertically to at least partially surround the cartridge, the sheet of material and cartridge being supported in a frame.

2. A refill according to claim 1, wherein the sheet of material comprises the porous pad, a rear panel and two side panels which are folded inwardly along the vertical fold lines towards one another from opposite sides of the rear panel.

3. A refill according to claim 2, wherein the porous pad is foldably attached to a bottom edge of the rear panel along a fold line which, in use, is substantially horizontal.

4. A refill according to claim 2, wherein the porous pad is attached to one or both bottom edges of the side panels along a fold line which, in use, is substantially horizontal.

5. An air freshener device having a refill according to claim 1 any one of the preceding claims.

6. A refill according to claim 1 any one of the preceding claims, wherein the folded sheet is retained in the frame by the resilience of the folded structure.

7. A refill according to claim 1 any one of the preceding claims, wherein the frame is non-foldable.

8. A refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded to at least partially surround the cartridge, the sheet of material and cartridge being supported in a frame; wherein the sheet of material is folded to meet the cartridge towards the end of the cartridge opposite to the end from which the composition is released.

9. A refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded to at least partially surround the cartridge, the sheet of material and cartridge being supported in a frame; wherein the cartridge is retained in at least one pair of resilient jaws in the frame.

10. A refill according to claim 9, wherein the cartridge is retained in two pairs of resilient jaws, the pairs being spaced along the length of the cartridge.

11. A method of manufacturing a refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded to at least partially surround the cartridge, the sheet of material and cartridge being supported in a frame; the method comprising the steps of: folding the porous pad over a mandrel; removing the mandrel from the folded pad; and inserting the folded pad into the frame, so that the folded pad is retained within the frame by the resilience of the folded structure.

12. A method according to claim 11, further comprising the step of inserting the cartridge into the frame from the opposite direction to the direction in which the pad is inserted on the mandrel.

13. A method according to claim 12, wherein the cartridge is retained in at least one pair of resilient jaws in the frame.

14. A method of making a refill for a liquid air freshener or insecticidal device comprising a cartridge containing a volatile liquid composition to be evaporated and a porous pad positioned, in use, to receive the liquid composition as it is slowly released from the cartridge; wherein the porous pad is a part of a sheet of material which is folded to at least partially surround the cartridge, the sheet of material and cartridge being supported in a frame; the method comprising the steps of: inserting the folded pad into the frame in a first direction; and inserting the cartridge into the frame in a second direction in which is opposite to the first direction.

15. A method according to claim 14, wherein the cartridge is retained in at least one pair of resilient jaws in the frame.

16. (canceled)
